(19) United States
(12) Patent Application Publication
(10) Pub. No.: US 2002/0069421 A1
(43) Pub. Date: Jun. 6, 2002

(54) Large scale expression and purification of recombinant proteins

(76) Inventors: Calvin C. Hale, Columbia, MO (US); Elmer M. Price, Columbia, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/901,419

(22) Filed: Jul. 9, 2001

Related U.S. Application Data
(60) Provisional application No. 60/218,125, filed on Jul. 13, 2000.

(51) Int. Cl. A01K 67/033; C12N 15/866

(52) U.S. Cl. 800/4; 800/8; 435/456

(57) ABSTRACT

The present invention is directed toward a method to produce recombinant fusion proteins in large quantities that are both highly homogenous and biologically active. In particular, the invention relates to a method for producing recombinant fusion proteins in a larvae expression system. The recombinant fusion protein is then purified from the larvae by an affinity tag fused to the protein via affinity chromatography.

### CROSS REFERENCE TO RELATED APPLICATION

This application claims priority from Provisional Application Serial No. 60/218,125 filed on Jul. 13, 2000, which is hereby incorporated by reference in its entirety.

### FIELD OF THE INVENTION

The current invention relates to a method to produce recombinant proteins. More particularly, the method provides a means to produce recombinant proteins by employing a larvae expression.

### BACKGROUND OF THE INVENTION

The ability to produce and subsequently purify a large quantity of recombinant protein in an efficient manner and at a relatively affordable cost from a host organism is a hallmark of recombinant technology. This is particularly true if the resulting protein has biological activity and can be purified to a high degree of homogenicity. The ability to achieve these goals is largely influenced by both the type of protein expressed and the host organism selected for this expression.

The type of protein, as stated above, dramatically impacts the efficiency, yield and cost of recombinant protein production. Broadly stated, proteins may be classified in two groups: soluble proteins and membrane proteins. Soluble proteins are proteins that are not integrally associated with a cell membrane or other structure and are generally free in solution. Because they are free in solution, soluble proteins may be readily purified in large quantities that are typically biologically active. Membrane proteins, on the other hand, are a part of or closely associated with a cell membrane and therefore, are typically not free in solution. This class of proteins, accordingly, are exceptionally more difficult to purify relative to soluble proteins, because prior to purification, their association with the lipid bilayer must be disrupted so that they become solubilized. While membrane proteins can generally be solubilized by detergents, these detergents often result in protein denaturation. As a consequence, a major obstacle encountered purifying membrane proteins is the inability to obtain large quantities of biologically active protein.

Additionally, the type of host organism selected also impacts the efficiency, yield and cost of recombinant protein production. A number of prokaryotic expression systems have been employed with varying degrees of success. The most common prokaryotic host is the bacterium Escherichia coli. There are many advantages to utilizing this expression system. First, in E. coli cells plasmids are frequently expressed in multiple copies, resulting in high expression of the foreign protein. Next, these cells divide rapidly, so that it is possible to purify large quantities of the recombinant protein in a short period of time. Finally, this method of protein production is relatively inexpensive. There are, however, serious drawbacks to selecting E. coli, or any prokaryotic system for that matter, to express eukaryotic proteins. This is because a large number of eukaryotic proteins require post-translational modifications in order to properly fold or function. Prokaryotic hosts do not possess cellular mechanisms to perform these modifications. And often times, the resulting proteins are unusable for functional or structural studies. This becomes a particularly critical limitation when the protein expressed is a membrane protein because, as stated above, membrane proteins are especially difficult to purify in large quantities that are biologically active.

To overcome these shortcomings, several eukaryotic expression systems have been developed. For example, Saccharomyces cerevisiae (yeast) was the first, and remains the most commonly employed eukaryotic expression system because its genome and physiology have been extensively characterized. These eukaryotic hosts offer several advantages over their prokaryotic counterparts. One such advantage is that they have an intracellular environment that is more conducive for correct folding of eukaryotic proteins. Additionally eukaryotic hosts, unlike prokaryotic hosts, have the ability to glycosylate proteins, which is important for both the stability and biological activity of the protein. Yeast are not always the optimal expression system, however, for the large-scale production of heterologous proteins because of plasmid loss during scale-up, hyperglycosylation, and low protein yields. This aspect, again, is a particularly critical limitation when the protein expressed is a membrane protein.

A recent alternative eukaryotic expression system employs insect cells transfected with a baculovirus as hosts for recombinant protein expression. In this system, the protein can be expressed at high levels once the virus infects the insect cell. Not only do these hosts express proteins at high levels, but the insect cells are particularly valuable host organisms due to their ability to accomplish most eukaryotic post-translational modifications including phosphorylation, N- and O-linked glycosylation, acylation, disulfide cross-linking, oligomeric assembly and subcellular targeting.

The use of insect cells as hosts for protein production, however, does have a serious drawback. This is because at the molecular level, manipulation of baculoviruses can present a significant challenge. A baculovirus genome comprises approximately 130 kb of DNA. Thus, making it too large for conventional plasmid cloning techniques. A common solution to this problem has been to introduce foreign genes by homologous recombination. This recombination, however, has a very low success rate and often results in screening countless numbers of clones in order to identify a clone that has successfully undergone proper recombination. Accordingly, protein production in insect cells is generally demanding and may be inefficient.

To overcome these obstacles, recent studies have addressed the issue of efficient, low-cost production of recombinant protein in baculovirus-infected insect larvae. In one such study, human adenosine deaminase (ADA), an essential enzyme in the purine salvage pathway, was produced in baculovirus-infected cabbage looper larvae (Trichoplusia ni) (Medin et al., Proc. Natl. Acad. Sci. USA, Vol. 87, pp.2760-2764). The resulting recombinant protein had a specific activity and structure comparable to native ADA. Additionally, the purification resulted in a high yield of protein, demonstrating that the use of baculovirus-infected insect cells for protein production may be inexpensive and rapid. One drawback to this study, however, is that it only addressed the issue of large-scale production of soluble recombinant proteins. No information was provided regarding the feasibility of producing membrane proteins in a larvae expression system.

Another study, however, examined the feasibility of a larvae expression system for the production of membrane proteins (Hale et al., Protein Expr Purif., February 1999; 15(1):121-126). In this study, recombinant bovine NCX1, a membrane transport protein, in baculovirus was used to infect cabbage looper larvae (Trichoplusia ni). Vesicle membranes isolated form the larvae proved to contain high levels of recombinant NCX1 protein, whose specific activity and structure were similar to native NCX1. This method, while promising, however, has a significant limitation. While Hale et al. were able to obtain large amounts of active protein, all of the protein was confined to larval vesicle membranes. Accordingly, their techniques does not provide a means for the amenable purification of recombinant membrane protein out of the larval vesicles. Without this capability, their method has little practical significance.

Accordingly, a need exists to devise improved methods for purifying recombinant membrane proteins. Ideally, this method would result not only in the production of a large quantity of the protein at a relatively affordable cost, but would also yield a protein with biological activity and structure comparable to the native protein.

### SUMMARY OF THE INVENTION

Among the several aspects of the invention, therefore, is provided a method for producing a recombinant protein in an insect larvae expression system, comprising infecting larvae with a vector containing a nucleic acid sequence encoding a recombinant fusion protein that includes an affinity tag, wherein the recombinant protein is expressed in the larvae and purifying the recombinant protein from the larvae by affinity chromatography.

Another aspect provides a method for identifying the physical characteristics of a recombinant fusion protein, wherein the protein is produced by the method comprising the insect larvae expression system.

### BRIEF DESCRIPTION OF THE DRAWINGS

These and other features, aspects, and advantages of the present invention will become better understood with regard to the following description, appended claims and accompanying figures where:

Table 1 depicts the results of poly (His) affinity purification via a nickel affinity column of recombinant NCX1. The protein was purified in accordance with the procedures set-forth in the Materials and Methods portion of the Example section. Column protein recovery and affinity purified recombinant NCX1 are compared.

FIG. 1 depicts SDS-PAGE and immunoblot analyses of NCX1-his in larvae membrane vesicles. Trichoplusia ni 4th instar larvae were infected with the NCX1-his construct and used to prepare membrane vesicles as described in the Materials and Methods portion of the Example section. Approximately 30 μg of vesicle protein was applied to each lane. The positions of the 120 and 70 kDa form of NCX1-his are indicated. A. Coomassie blue stained SDS-PAGE under reducing [lane 1] and nonreducing [lane 2] conditions. B. Immunoblot of larvae vesicles probed with NCX1 antibody. Lane 1—membrane vesicles from uninfected larvae, reducing conditions (control); Lane 2—membrane vesicles from infected larvae, nonreducing conditions; Lane 3—membrane vesicles from infected larvae, reducing conditions.

FIG. 2 depicts NCX transport in NCX 1-his larvae membrane vesicles. Larvae membrane vesicles containing NCX1-his were subjected to NCX1 activity as previously described (Hale, et al., 1999). At time=0, membrane vesicles were diluted 5-fold into an isotonic KCl solution containing ⁴⁵Ca²⁺. Transport was terminated at the indicated times (•). Arrow: Following 30 s of Na⁺-dependent ⁴⁵Ca²⁺, of Na⁺-dependent ⁴⁵Ca²⁺ efflux was initiated by adjusting the external solution to 200 mM NaCl (o).

FIG. 3 depicts electrophoretic analysis of NCX1 affinity column chromatography. NCX1-his in larvae membrane vesicles was solubilized in a 2% sodium cholate buffer and subjected to chelated Ni⁺ affinity column chromatography as described in the Materials and Methods portion of the Example section. A. SDS-PAGE visualized via silver stain. Lane 1—sodium cholate solubilized larvae membrane proteins (column load); Lane 2—column flow through (unbound material); Lane 3—column wash; Lane 4—eluted proteins. B. Immunoblot analysis of eluted proteins (lane 4).

### ABBREVIATIONS AND DEFINITIONS

To facilitate understanding of the invention, a number of terms and abbreviations as used herein are defined below:

“Biological activity substantially the same as the native form of the protein” shall mean that the recombinant fusion protein produced by the method of the current invention is capable of performing substantially the same function as the native form of the protein.

“Structurally substantially the same as the native form of the protein” shall mean that the recombinant fusion protein produced by the method of the current invention exhibits substantially the same tertiary and quaternary structure as the native form of the protein.

“Substantially pure” or “Isolated” are used herein interchangeably, when referring to proteins and polypeptides, and denotes those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure protein will make up from about 75 to about 90% of the total protein. Preferably, the protein will make up greater than about 90%, and more preferably, greater than about 95% of the total protein in the composition, even more preferably the protein will make up greater than about 97% of the total protein in the composition.

“Homogenous or Purified Sample” are used interchangeably and mean a sample or composition wherein the recombinant fusion protein of the present invention is the dominant protein present is said sample or composition. Preferably, the protein will make up greater than about 90%, and more preferably, greater than about 95% of the total protein in the composition, even more preferably the protein will make up greater than about 97% of the total protein in the composition.

“Recombinant form of the protein” shall mean a non-native protein derived by recombinant means or a native protein with an altered amino acid sequence.

“Native form of the protein” shall mean the form of protein naturally occurring in the intact cell.

“Recombinant Nucleic Acid” is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, for example, products made by transforming cells with any unnaturally occurring vector is encompassed, as are nucleic acids comprising sequences derived using any synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design.

“Recombinant Fusion Protein” means the protein resulting from the expression product of two fused nucleic acid sequences.

“Polynucleotide” and “oligonucleotide” are used interchangeably and mean a polymer of at least 2 nucleotides joined together by phosphodiester bonds and may consist of either ribonucleotides or deoxyribonucleotides.

“Sequence” or “nucleic acid sequence” means the linear order in which monomers occur in a polymer, for example, the order of amino acids in a polypeptide or the order of nucleotides in a polynucleotide.

“Soluble Protein” shall mean, as used herein, any protein that is not an integral part of or closely associated with a cell membrane.

“Membrane Protein” shall mean any protein that is normally an integral part of or closely associated with a cell membrane.

“Affinity Tag or Label” are used herein interchangeably and mean any polypeptide sequence that confers a means to purify the recombinant fusion protein to which said affinity tag is fused when the recombinant protein is purified by affinity chromatography.

“Operably linked” means a unit of coordinated and regulated gene activity by means of which the control and synthesis of a protein is determined. It consists of a DNA region encoding a protein together with one or more regions that regulate transcription, such as a promoter.

“Instar stage of development” shall mean a method to characterize the growth and development of larvae at different stages of their life cycle. For purposes of this invention a first, second, third, fourth and fifth instar stage of development classification system is utilized. The classification system is described in Coudron et al., (1990) Arch. Insect Biochem. Physio. 13:83-94.

“Early fourth instar stage of development” shall mean the time in the growth cycle of the larvae when the exuvium of the third instar slips off the anterior end, but still remains attached to the abdominal segments of the larvae.

NMR=nuclear magnetic resonance

CD=circular dichroism

kDa=kilo dalton

SDS-PAGE=sodium dodecyl sulfate polyacrylamide gel electrophoresis

NCX1=cardiac sodium-calcium exchange protein

Na-K ATPase=sodium-potassium exchange protein

CFTR=cystic fibrosis transmembrane conductance regulator

### DESCRIPTION OF THE PREFERRED EMBODIMENT

Applicants have discovered a method to purify recombinant fusion proteins utilizing an insect larvae expression system. The method comprises infection of insect larvae with a vector that has a nucleic acid sequence encoding a recombinant fusion protein of interest with an attached affinity tag. The recombinant fusion protein is then expressed and purified from the larvae by affinity chromatography. This method provides a means to produce large quantities of active recombinant protein resulting in a virtually homogenous sample.

The present invention employs the use of recombinant technology to produce large quantities of a desired recombinant fusion protein. The recombinant protein construct utilized in the invention results from the fusion of two genes. The first gene encodes a protein desired for large scale production (“target protein”) and the second protein encodes an affinity tag used to purify the target protein. The target protein is not limited to any particular class of proteins and may include both soluble and membrane proteins. Preferably the target protein will be a membrane protein. Again, the membrane protein is not limited to any particular class of membrane proteins and may include transport, channel forming, receptor, junctional, cytoskeletal and other membrane associated proteins. In a preferred embodiment, the present invention is used to produce the transport proteins NCX1, sodium-iodide transporter, sodium-phosphate transporter, Na—K ATPase, and the channel forming protein CFTR. Another embodiment of the invention encompasses producing the junctional protein conexin 32 and the protein prostate specific membrane antigen. In yet another embodiment, the method may be employed to produce the sodium phosphate co-transporter from kidney.

The affinity tag of the present invention is not limited to any particular sequence or feature other than providing a means to purify the target protein from the larvae to a high degree of homogenicity. Thus, any class of affinity tag commonly known to those skilled in the art may be employed.

In one embodiment, the affinity tag is a metal chelating peptide. In general, preferred metal chelating peptides include His-X wherein X is, for example, Gly, His, Tyr, Gly, Trp, Val, Leu, Ser, Lys, Phe, Met, Ala, Glu, Ile, Thr, Asp, Asn, Gln, Arg, Cys or Pro as described more fully in Smith et al. (1986) U.S. Pat. No. 4,569,794. Preferably, the metal chelating peptide includes (His-X)ₙ wherein X is Asp, Pro, Glu, Ala, Gly, Val, Ser, Leu, Ile or Thr and n is at least 3 as described more fully in Sharma et al. (1997) U.S. Pat. No. 5,594,115. More preferably, the metal chelating peptide includes a poly(His) tag of the formula (His)_{y} wherein y is at least 2-6 as described more fully in Dobeli et al. (1994) U.S. Pat. No. 5,310,663. The poly (His) tag allows a protein to which it is attached to be purified based upon its affinity for a charged metal immobilized to a surface. When the poly(His) tag is utilized any number of His residues may be included in the affinity tag to the extent that the tag affords purification of the target protein to the desired degree of homogenicity.

In another embodiment, the affinity tag comprises a biotin capture system. For example, avidin or streptavidin tags may be employed as described more fully in Skerra et al. (1996) U.S. Pat. No. 5,506,121 . In general, the avidin or streptavidin tag allows a protein to which it is attached to be purified based upon its affinity for biotin.

In a further embodiment, the affinity tag comprises an enzymatic capture system. Such systems are more fully described in Smith (1997) U.S. Pat. No. 5,654,176. For example, glutathione-S-transferase belongs to this class of affinity label. The glutathine-S-transferase tag allows a protein to which it is attached to be purified based upon its affinity for its substrate.

In a further embodiment, an immunogenic capture system is employed. Such systems include an antigenic sequence (and optionally a cleavage site) such as the DYKDDDK sequence disclosed in Hopp et al (1991) U.S. Pat. No. 5,011,912, or Hopp et al (1987) U.S. Pat. No. 4,703,004 or the DLYDDDK sequence. The immunogenic tag allows the protein to which it is attached to be purified based upon its affinity for an antibody.

The affinity tag is preferably fused to the target protein in a manner such that the biological activity and structure of the target protein are not significantly impacted. Hence, the affinity tag may be placed on either the C-terminus or N-terminus of the target protein to the extent that biological activity and structure of the target protein are not impacted. One possessing ordinary skill in the art can readily position the affinity tag so as to minimize the impact to activity and structure of the target protein. For example, a preferred embodiment of the present invention employs a 6 residue poly (His) affinity tag fused to the C-terminus of a recombinant NCX1 protein. The poly(His) tag, as detailed below in the examples, does not impact either the biological activity or the structure of the recombinant NCX1 protein and provides a means to purify the protein to near complete homogenicity.

The construction of the recombinant fusion protein of the present invention may be performed by any generally known method. Additionally, the gene encoding the target protein may be subcloned from an organism using a variety of procedures known to those skilled in the art and detailed in, for example, Sambrook et al., Molecular Cloning, A Laboratory Manual, 2nd ed., Cold Spring Harbor Laboratory Press, (1989) and Ausabel et al., Short Protocols in Molecular Biology, 3rd. ed., John Wiley & Sons (1995). In a preferred method, full length cDNA encoding the target protein is subcloned into viral DNA as detailed in Hale et al., (1999) Protein Expression and Purification 15:121-126. The resulting construct is then inserted into a bacterial plasmid vector and subjected to site-directed mutagenesis such that a poly(His) tag is added to the target protein at the desired location on such protein. The bacterial plasmid vector selected for this step is not critical to the invention; however, the plasmid preferably is easy to manipulate and provides a means to efficiently amplify the recombinant fusion protein construct. The method of inserting the construct into the vector is not critical to the invention and may be accomplished by any means generally known in the art. Preferably, the sequence is inserted into an appropriate endonuclease restriction site(s) in the vector. Additionally, site directed mutagenesis may be performed employing a number of generally known techniques as detailed in, for example, Sambrook et al., Molecular Cloning, A Laboratory Manual, 2nd ed., Cold Spring Harbor Laboratory Press, (1989), and Ausabel et al., Short Protocols in Molecular Biology, 3rd. ed., John Wiley & Sons (1995). Upon its amplification, the resulting construct encoding the recombinant fusion protein may be excised from the vector by appropriate restriction digestion. Preferably, the construct encoding the recombinant fusion protein is subjected to restriction mapping and sequencing in order to ensure that said construct has the correct nucleic acid sequence.

The construct encoding the recombinant fusion protein of the present invention is then inserted into a vector capable of infecting insects. The invention is not limited to any particular type of vector. However, the vector utilized in the expression system preferably will be capable not only of infecting insect cells, but also will preferably infect larvae, and typically will be capable of subsequently directing such cells or larvae to express the recombinant fusion protein encoded by said vector.

Thus, in a preferred method of the present invention a baculovirus expression system is utilized. The salient features of a baculovirus expression system include the co-transformation into insects (cells of larvae) of a baculovirus transfer vector along with complete intact viral genomic DNA. A typical baculovirus transfer vector includes: sequences to allow propagation in bacteria, a polyhedrin gene promoter, the polyhedron mRNA polyadenylation signal, and sequences that, in the virus, flank both ends of the polyhedrin gene. The construct encoding the recombinant fusion protein to be expressed is inserted into the vector such that it is adjacent and operably linked to the polyhedrin promoter (or other suitable promoter in a baculovirus system). Once the DNA is inside the insect, homologous recombination can take place whereby the polyhedrin gene on the viral genomic DNA is replaced with the construct encoding the recombinant fusion protein. This recombination results in the generation of a modified virus with the recombinant fusion protein. A resulting mixture of plaques with and without transfer vector integration occur. However, plaques with the modified virus are readily identifiable based on visual inspection. The recombinant fusion protein may be excised from the modified virus by restriction digestion and subjected to DNA sequencing in order to ensure said virus contains the sequence of the recombinant fusion protein. This vector is then ready for injection into larvae. The description of specific components of the baculovirus expression system set-forth above, such as the polyhedrin gene or promoter, is not critical for the present invention. For example, one skilled in the art could readily employ a baculovirus system with different components that would equally accomplish the features of the invention.

The viral vector encoding the recombinant fusion protein is injected into larvae. The injection procedure and rearing of the larvae can be accomplished by any generally known methods as detailed, for example, in Medin et al., (1990) Proc. Natl. Acad. Sci. 87:2760-74. The choice of larvae species is not a critical feature of the present invention. In a preferred embodiment cabbage looper larvae (Trichoplusia ni) are utilized. Additional larvae species that may be utilized in other embodiments include, but are not limited to Pflutella xylostella, alfalfa looper, Idalima leonora and Periscepta polysticta. Additionally, larvae are preferably injected when they are at the early fourth instar stage of development. This stage optimizes both size for ease of injection and the amount of recombinant fusion protein expressed. In another embodiment, larvae in the first, second, and third instar stage of development may be injected. However, due to their small size these stages of development are less preferable than the early fourth instar stage of development. Larvae past the early fourth instar stage of development are preferably not used as recombinant fusion proteins produced during this stage are subject to a high post translational error rate. The instar stages of larvae development are fully described in Caldron et al., (1990) Arch. Insect Became. Physic. 13:83-94.

In a preferred embodiment, the larvae are allowed to develop for precisely 3-3 ½ days post infection prior to harvesting the recombinant fusion protein. This allows for maximum expression of the recombinant fusion protein. In another embodiment, the larvae may be allowed to develop for 1 or 2 days post infection prior to harvesting the recombinant fusion protein. However, such harvest at this stage results in expression of a minimal amount of recombinant fusion protein. The larvae preferably are not allowed to develop more than 4 days post infection prior to harvest of the recombinant fusion protein as the resulting recombinant protein is subject to a high mutation rate. The infected larvae may be stored at −70° C. prior to use.

The recombinant fusion protein may be isolated from the larvae by affinity chromatography or any other method generally known in the art. In a preferred method, a fraction containing the recombinant fusion protein is isolated from the larvae by differential and gradient centrifugation. The procedure of differential and gradient centrifugation involves homogenizing the larvae in an appropriate buffer and then subjecting the homogenized product to a series of centrifugation steps wherein different speeds and times are employed at each said centrifugation step. Each step results in a fraction that is more enriched with the recombinant fusion protein. The procedure to be employed for the centrifugation process will vary depending on the particular characteristics of the recombinant fusion protein. For example, soluble proteins will be in a different fraction than membrane proteins and organelle membrane proteins will be in a different fraction than plasma membrane proteins. One possessing ordinary skill in the art of protein purification can readily develop a protocol tailor made to optimally isolate protein fractions containing any particular class of recombinant fusion protein and also any particular recombinant protein. Such procedure can be developed and optimized by checking for the physical presence of the recombinant fusion protein in the fraction at each step of centrifugation by subjecting the fraction of interest to Western Blot analysis. Additionally, the activity of the recombinant fusion protein in the fraction can also be monitored at each step of centrifugation. In addition to differential and gradient centrifugation, other generally known methods may be employed in order to isolate a fraction containing the recombinant fusion protein.

The recombinant fusion protein may be further purified from the isolated fraction by methods such as affinity chromatography, size exclusion chromatography or ion exchange chromatography. In a preferred embodiment, affinity chromatography is utilized. The steps employed in the affinity chromatography will be driven by the type of affinity tag fused to the recombinant protein. For example, when the affinity tag is avidin or streptavidin, the recombinant protein may be purified from the fraction by passing the fraction through a column containing immobilized biotin. The biotin specifically binds a recombinant protein possessing an avidin/streptavidin tag based upon the affinity of biotin for avidin/streptavidin (biotin binds to avidin/streptavidin in a non-covalent manner). Hence, any protein in the fraction not possessing the avidin/streptavidin tag will pass through the column. The non-covalent association of biotin and avidin may then be disrupted by application of an appropriate buffer to the column. The resulting recombinant fusion protein is, at that point, purified to a high degree of homogenicity. Additionally, if the recombinant protein to be purified is a membrane protein then preferably a detergent is utilized in the buffer to solubilize the protein. Preferably, non-ionic detergents are employed for such solubilization as they do not interfere with purification by affinity chromatography whereas ionic detergents may interfere with such purification. In a preferred embodiment, sodium cholate is utilized. Another preferred method of the invention encompasses further purifying the protein after affinity purification by dialysis. The dialysis may be performed according to any generally known method.

After its purification from the protein fraction by affinity chromatography, the recombinant fusion protein is in a highly pure fraction. However, the recombinant fusion protein still possesses the affinity tag. Depending on the desired use of the recombinant protein, the affinity tag may be removed by any method known in the art. In a preferred method, the affinity tag is removed by a protease such as an enterokinase possessing cleavage specificity at the appropriate site on the recombinant fusion protein. In yet another method, the protease is covalently immobilized to a bead, such as sepharose.

In order to determine whether the recombinant protein possesses biological activity after being subjected to the purification process employed by the invention, the recombinant protein may be utilized in an activity assay. The activity assay will be different for each particular recombinant fusion protein. One skilled in the art can determine an appropriate activity assay for the particular recombinant fusion protein. In general, upon development of such an activity assay, both the native form of the protein and the recombinant form of the protein are employed in the activity assay wherein both are subjected to the same assay conditions. The relative specific activity of the native versus the recombinant form is then compared. Preferably, the recombinant fusion protein will have substantially the same biological activity relative to the native protein. However, the acceptable level of specific activity possessed by the recombinant protein will vary greatly depending upon its intended application. For example, if the recombinant protein is to be utilized for the purpose of protein crystal formation, then the recombinant protein ideally exhibits a very high level of specific activity relative to the native form of the protein. However, if the intended purpose of the recombinant fusion protein is for sequencing, then a lower level of specific activity relative to the native form is tolerable.

The method for producing a recombinant protein according to the present invention, as exemplified by the example delineated below, provides a means to produce large quantities of an active recombinant protein in a highly purified form. The purified recombinant protein may then be utilized in a number of different applications.

In one such application, the recombinant protein produced by the method of the current invention may be employed to biophysically analyze said recombinant protein. For example, many methods for physically characterizing proteins require large quantities of highly active protein. These methods include but are not limited to crystallography, NMR, and CD. Hence, the method of the current invention provides a means to purify sufficient quantities of highly active recombinant protein that may be employed in any of these applications.

In yet another application, the recombinant protein produced by the method of the current invention may be included as a part of a pharmaceutical, nutritional, drug or vaccine composition. Those of ordinary skill in the art of preparing pharmaceutical formulations can readily formulate pharmaceutical compositions having recombinant fusion proteins produced by the method of the invention using known excipients (e.g. saline, glucose, starch, etc.). Similarly, those of ordinary skill in the art of preparing nutritional formulations can readily formulate nutritional compositions having recombinant fusion proteins produced by the method of the invention. And those of ordinary skill in the art of preparing food or food ingredient formulations can readily formulate food compositions or food ingredient compositions having recombinant fusion proteins produced by the method of the invention.

In addition, those of ordinary skill in the art can readily determine appropriate dosages that are necessary to achieve the desired therapeutic or prophylactic effect upon oral, parenteral, rectal and other administration forms. Typically, in-vivo models (i.e., laboratory mammals) are used to determine the appropriate dosage to effect the desired result.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not imitative of the remainder of the disclosure in any way whatsoever.

### EXAMPLES

### Example 1

The following example details the successful implementation of the larvae expression system of the current invention. In this example, a recombinant membrane transport protein, NCX1, is produced in large quantities that are both highly active and pure.

### Materials and Methods

Generation of Baculovirus Construct

Bovine NCX1 cDNA was originally obtained in the vector pcDNA (Aceto et al., (1992)Arch. Became. Biosphys. 298:553-560). The full-length cDNA was excised from pcDNA and subsequently subcloned into Baculogold viral DNA as previously described (Hale et al., (1999) Protein Expression and Purification 15:181-126). The full-length cDNA was inserted into pBluescript and subjected to site-directed mutagenesis which resulted in the addition of 6 histidines to the NCX1 C-terminus. The mutated construct was subcloned into the baculovirus transfer vector pVL1392 for co-transfection with Bac 3000 (Invitrogen) in Sf9 cells. Plaque-pure recombinant baculovirus was prepared according to established procedures (Webb et al., (1990) Technique 2:173-178). Several plaques were picked in the initial isolation procedure. NCX1-his expressors were identified by immunoblot analyses. One of the plaques was chosen for scale-up and the resulting viral stock (NCX1-his-RVS) was used in all of the following experiments. The sequence of the NCX1 construct with the inserted poly(His) tag, as detailed above, is set forth as SEQ ID NO:1.

Infection of Cabbage Looper Larvae

Larvae (Trichoplusia ni) were reared and injected according to previously described methods (Medin et al. (1990) Proc. Natl. Acad. Sci. 87:2760-2764). Briefly, early fourth instar larvae were placed on ice for a minimum of 10 minutes or gassed with 100% CO₂ for 5 s which resulted in temporary immobilization. NCX1-RVS (approximately 5×10⁵ viral molecules in 4 μl aliquots) were injected into the larvae using a 28.5 gauge needle and a 100 μl Hamilton syringe. The injected larvae were returned to their media cup which was held at ambient temperature for 3 days, after which time the larvae were frozen at −70° C.

Vesicle Preparations

Membrane vesicles from Trichoplusia ni were prepared as previously described although the fresh weight of the starting material varied from 4-20 g. A standard preparation proceeded as follows: Frozen larvae (19-20 larvae, approximately 4 g total) were polytron homogenized (low setting; 20 s) in 100 ml of 250 mM sucrose, 20 mM MOPS adjusted to pH 7.4 with Tris (MOPS/Tris) and the following protease inhibitors: 1,000 K.I.U/L aprotinin, 340 nM leupeptin, 970 nM pepstatin A, and 190 μM phenylmethylsulfonyl fluoride (grinding buffer). The homogenate was subjected to a low speed centrifugation (1,000×g, 10 min, 4° C.). A layer of debris that formed on top of the supernatant was aspirated and discarded. The supernatant fluid (S1) was removed and saved. The pellet was resuspended in 100 ml of grinding buffer and further homogenized (polytron, 3×30 sec, medium setting). The homogenate was centrifuged at 10,000×g for 10 min, 4° C. The supernatant (S2) was saved and the pellet was subject to an additional round of homogenization and centrifugation (S3). Supernatants S1, S2, and S3 were pooled and centrifuged at 120,000×g, 45 min, 4° C. The resultant pellets were resuspended in 25 ml of 8% sucrose (w/v), homogenized with a Potter-Elvehjem tissue grinder, layered on a 36% sucrose pad, and subjected to gradient centrifugation at 180,000×g, 90 min, 4° C. A fluffy vesicle layer at the gradient interface was removed and diluted 4-fold in 160 mM NaCl, 20 mM MOPS/Tris, pH 7.4. Vesicles were pelleted at 204,000×g for 30 min. The pellets were resuspended in the above NaCl buffer (approximately 2-4 mg/ml), homogenized (Potter-Elvehjem tissue grinder), aliquoted, and stored at −70° C.

Chelated Ni²⁺ Affinity Column Chromatography

Polyhistidine tagged recombinant NCX1 protein was purified using a commercially available kit (HisTrap; Pharmacia Biotech). Larvae membrane vesicles (approximately 10 mg protein) were pelleted at 204,000×g for 30 min at 4° C. The pellet was resuspended and solubilized in 10 ml of column start buffer which consisted of 2% sodium cholate, 0.5 M NaCl, 10 mM imidazole, 20 mM sodium phosphate, pH 7.4 and maintained on ice with periodic mixing for 30 min. The solubilized preparation was loaded on to a 1 ml chelated Ni²⁺ affinity column. The column was washed with a minimum of 20 volumes of start buffer. Bound protein was eluted from the column with start buffer containing 500 mM imidazole. Fractions containing NCX1 protein were monitored by Western blot analysis. Soybean phospholipids (Associated Concentrates, Woodside, N.Y.; 25 mg/ml final concentration) were added to fractions containing NCX1 protein and incubated on ice for 15 min with periodic vortex mixing. Reconstitution into proteoliposomes was accomplished by detergent dilutions. Briefly, the phospholipid/detergent/protein mixture was rapidly diluted into 5 volumes of 160 mM NaCl buffer. The resultant sample was incubated on ice for 15 minutes with periodic vortex mixing followed by centrifugation at 204,000×g at 4° C. for 2 hr. The resultant proteoliposome pellet was washed in the 160 mM NaCl buffer by centrifugation (1 hr). The proteoliposome preparation was subjected to SDS-PAGE followed by Western blot analysis and NCX1 activity.

In the alternative, reconstitution into proteoliposomes was accomplished by dialysis. The material was dialyzed against 3-1 liter changes of 160 mM NaCl, 20 mM Mops/tris, pH 7.4 at 40° C. in dialysis tubing with a 100 kDa pore size. Following dialysis, the resulting proteoliposomes were washed by centrifugation as described above for 1 hr with the final pellet being resuspended in the 160 mM NaCl buffer.

NCX1 Activity Measurements

NCX1 activity was determined as previously described (Hale et al., (1999) Protein Expression and Purification 15:181-126 and Kleiboeker et al. (1992) J. Biol. Chem. 267:17836-17841). Transport was measured at 37° C. at the indicated time intervals in the presence of 12 μM ⁴⁵Ca²⁺. Experiments were repeated a minimum of two times on at least 2 different vesicle preparations. All points are the result of triplicate determinations. All transport data are corrected for Na+independent ⁴⁵Ca²⁺ influx passive influx (control).

Results

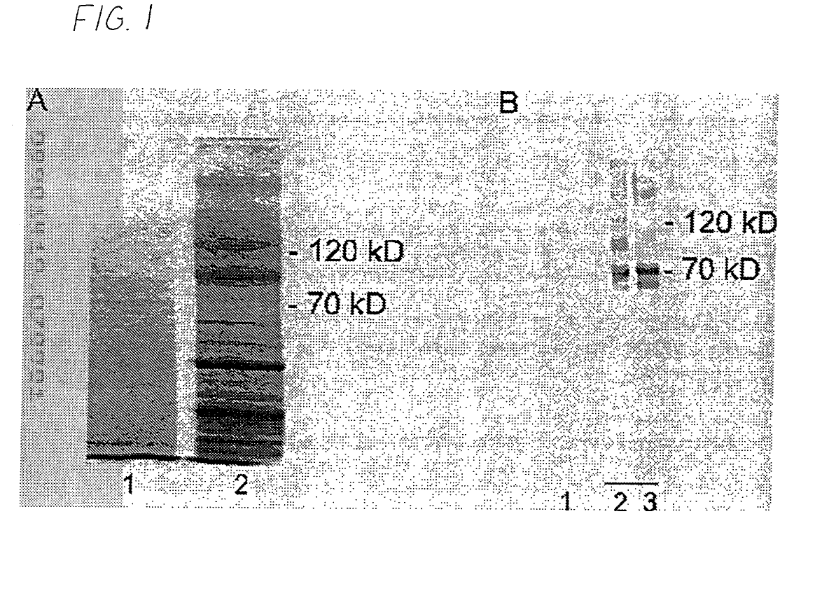

As previously reported, membrane vesicles from Trichoplusia ni infected with a baculovirus construct containing recombinant NCX1 had NCX1 activity that was mechanistically not different from activity observed in cardiac sarcolemmal vesicles (Hale et al., (1999) Protein Expression and Purification 15:181-126). In the previous study it was noted that the larval vesicle NCX1 protein, as observed by Western blot analysis, was essentially all 70 kDa. In contrast, NCX1 protein expressed in High Five cells (Trichoplusia ni cultured cells) existed as the 120 and 70 kDa form under nonreducing conditions. For the present study, a different baculovirus vector (Bac 3000; Invitrogen) was used because this vector has several viral proteins deleted including a protease and chitinase. As a result, under nonreducing conditions, the expressed NCX1-his protein observed in larvae vesicles was 120 and 70 kDa (FIG. 1). An additional band with an apparent Mr of 90 kDa also cross-reacted with the NCX antibody suggesting the presence of an intermediate proteolytic breakdown product. Under reducing conditions, the 70 kDa form of NCX1 was the predominant form. Upon closer examination, it was noted that the 70 kDa band existed as a doublet. This suggests that the expressed protein contained at least one proteolytic cleavage and that the 120 kDa form is held together by disulfide bridge interactions. The polyhistidine tag had no apparent affect on the protein's ability to migrate during SDS-PAGE. No bands were immunologically detected in control vesicle preparations.

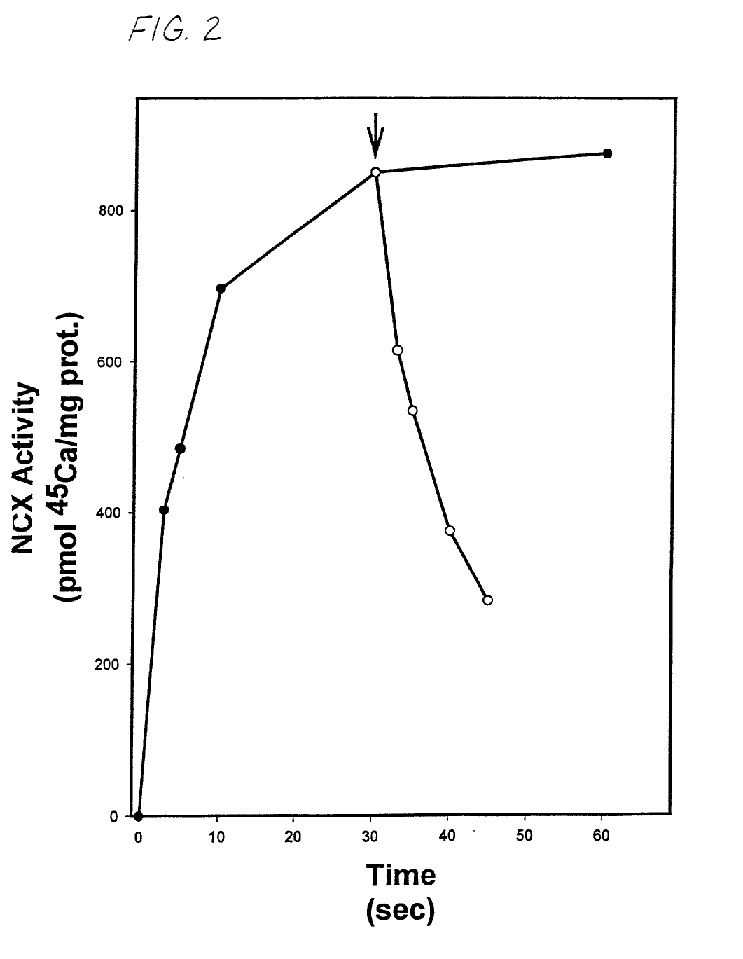

NCX1-his protein in larvae membrane vesicles was active and reversible as shown in FIG. 2. In these experiments, Na⁺-loaded membrane vesicles were diluted 20-fold into an isotonic solution of KCl creating an outwardly directed Na⁺ gradient. Under these conditions, NCX1-his catalyzed the influx of ⁴⁵Ca²⁺ into the vesicle lumen. No Na⁺-dependent ⁴⁵Ca²⁺ influx was observed in vesicles from control larvae membrane vesicles (not shown) as was previously reported (Hale et al., (1999) Protein Expression and Purification 15:181-126) further confirming the absence of endogenous exchange activity in this membrane subfraction. NCX1-his supported reverse mode exchange activity. The arrow in FIG. 2 indicates the addition of sufficient 2 M NaCl to raise the external solution Na⁺ concentration to 200 mM. Raising the extra vesicular Na⁺ concentration results in an inwardly directed Na⁺ gradient which, in the presence of NCX1-his, catalyzed ⁴⁵Ca²⁺ efflux from the vesicle lumen. Taken together, the data in FIGS. 1 and 2 indicate that a full-length, active NCX1 -his protein was expressed and present in the subfractionated larvae membrane preparation. The NCX1-his protein (and activity) was not observed in other subfractionated larvae membrane populations (not shown).

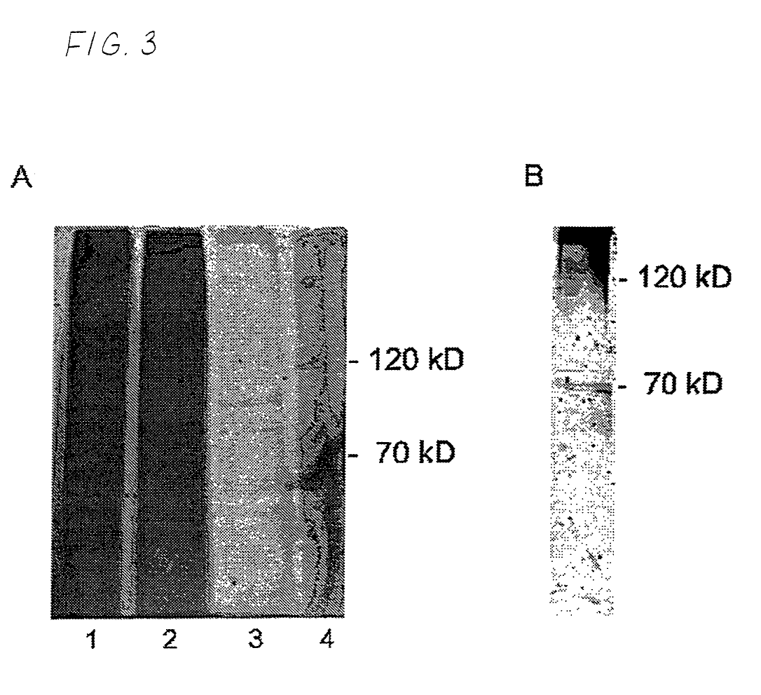

Larvae membrane vesicles containing NCX1-his were subjected to chelated Ni²⁺ affinity column chromatography as described in Materials and Methods. In these experiments, larvae vesicles were solubilized and extracted with sodium cholate, which has minimal ionic effects and has been successfully used in reconstitution experiments following column chromatography (Hale et al., (1984) Proc. Natl. Acad. Sci. 81:6569-6573). FIG. 3 shows how the column performed as judged by SDS-PAGE and immunoblot analyses. As shown in FIG. 3A, lanes 1 and 2, the majority of detergent solubilized membrane proteins extracted from the larvae vesicles were not bound or retained by the column. Extended washing of the column in start buffer essentially removed all larvae protein. Following the wash, bound proteins were eluted from the column. The eluted protein electrophoretic pattern shown in FIG. 3 demonstrates that the NCX1 protein was highly purified as the 120 and 70 kDa proteins were the major bands observed. The 90 kDa protein recognized by the NCX1 antibody (FIG. 1) was not observed in the final eluted fraction.

Chelated Ni²⁺ affinity column chromatography successfully purified recombinant NCX1-his protein but an important question remaining was whether or not NCX1-his retained a conformation that could catalyze Na⁺ and Ca²⁺ transport. This issue was addressed by reconstituting eluted NCX1-his protein into proteoliposomes comprised of soybean phospholipids. These results, including a summary of the purification are shown in Table 1.

TABLE 1
NCX1 AFFINITY COLUMN PURIFICATION

| | Larvae Vesicle Protein | |
|---|---|---|
| Sample | (mg) | % Column Load |
| Column Load | 12.27 ± 3.7 | 100 |
| Flow Through | 9.44 ± 3.2 | 77 |
| Wash | 1.58 ± 0.3 | 13 |
| Flow Through + Wash | 11.02 | 90 |
| Elution | 0.63 ± 0.2 | 5 |

| | NCX Specific Activity | |
|---|---|---|
| | (nmol ⁴⁵Ca/mg prot./sec) | Fold Purification |
| Larvae Vesicles | 0.042 ± 0.01 | — |
| Reconstituted | 0.362 ± 0.06 | 8 |
| Proteoliposomes | | |
| (from elution) by detergent | | |
| dilution | | |
| Reconstituted | 0.362 ± 0.06 | 13.4 |
| Proteoliposomes | | |
| (from elution) by dialysis | | |

Table 1 summarizes the combined results and performance of several typical affinity column purifications. Based upon the results shown in Table 1, it appears that recombinant NCX1-his comprised as much as 5% of the membrane proteins in the light larvae vesicle fraction. Affinity column purification and reconstitution by detergent dilution yielded a 8-fold increase in NCX1 specific activity. Affinity column purification and reconstitution by dialysis, on the other hand, yielded a 13.4-fold increase in NCX1 specific activity. In one purification experiment, solubilized membrane vesicle proteins obtained from 1,500 larvae were applied to the affinity column. The yield of affinity purified NCX1 protein was approximately 3 mg. Crystal screening trials using purified NCX1 protein were then initiated.

### Example 2

Conexin 32 is a member of a family of membrane proteins that form various junctions between cells. Conexin 32 is specifically found in mammalian heart.

Recombinant conexin 32 was expressed in the larvae expression system in accordance with the general guidelines set forth in example 1 above. The resulting expression was compared to that expressed in cell culture. Both expressions showed a characteristic laddering effect on Western blot analysis that results from formation of dimers and trimers. The larvae expressed protein was produced at an increase of nearly 100-fold higher than in cell culture, based on equal protein loads on gels. The larvae expressed protein, however, did show signs of proteolytic degradation as the apparent molecular weight of the bands observed was reduced compared to the protein expressed in cell culture. Nevertheless, the fact that the protein was in much higher abundance and capable of forming the characteristic laddering, makes the expression of this protein in the larvae expression system advantageous.

In view of the above, it will be seen that the several objectives of the invention are achieved and other advantageous results attained.

What is claimed is:

1. A method for producing a recombinant protein in an insect larvae expression system, the method comprising:
(a) infection of larvae with a vector that has a nucleic acid sequence that encodes a recombinant fusion protein with an affinity tag wherein the recombinant protein is expressed in the larvae; and
(b) purification of the recombinant protein from said larvae by affinity chromatography.

2. The method of claim 1 wherein the recombinant fusion protein is a membrane fusion protein.

3. The method of claim 1 wherein the affinity tag is selected from the group consisting of poly(His), avidin, biotin, antibody, streptavidin and an antigenic amino acid sequence.

4. The method of claim 3 wherein the affinity tag is poly(His).

5. The method of claim 1 wherein the vector is a baculovirus.

6. The method of claim 1 wherein the larvae are infected with the vector when the larvae are in the first, second, third, or fourth instar stage of development.

7. The method of claim 1 wherein the larvae are in the early fourth instar stage of development.

8. The method of claim 1 further comprising isolation of a protein fraction from the larvae wherein the fraction contains the recombinant fusion protein with the affinity tag.

9. The method of claim 8 wherein the fraction is isolated from the larvae by differential and gradient centrifugation.

10. The method of claim 9 further comprising isolation of the fraction by chromatography performed after the step of differential and gradient centrifugation.

11. The method of claim 1 further comprising removal of the affinity tag from the recombinant fusion protein.

12. The method of claim 2 wherein the recombinant membrane fusion protein is selected from the class of proteins consisting of transport, channel forming, receptor, junctional, cytoskeletal, and other membrane associated proteins.

13. The method of claim 12 wherein the recombinant membrane protein is a transport protein.

14. The method of claim 13 wherein the transport protein is NCX1 or the Na-K ATPase.

15. The method of claim 12 wherein the recombinant membrane protein is a channel forming protein.

16. The method of claim 15 wherein the channel forming protein is CFTR.

17. The method of claim 12 wherein the recombinant membrane protein is a junctional protein.

18. The method of claim 17 wherein the junctional protein is conexin 32.

19. The method of claim 1 wherein the recombinant fusion protein has biological activity substantially the same as the native form of the protein.

20. The method of claim 1 wherein the recombinant fusion protein has substantially the same structure as the native form of the protein.

21. A method for identifying the physical characteristics of a recombinant fusion protein wherein the protein is produced by the method of claim 1.

22. The method of claim 21 wherein the physical characteristics are determined by a procedure selected from the group consisting of crystallography, NMR, and CD

23. The method of claim 22 wherein the procedure is crystallography.


SEQUENCE LISTING (2 sequences)

SEQ ID NO: 1 - DNA, 4087 bp, Bos taurus
  CDS (268)..(3180)
  sig_peptide (268)..(363)
  misc_feature (3178): A Poly (H) affinity tag comprising 6 His residues have been inserted at the C-Terminus end of the coding region of the protein
gaattcgggagaagccatcaccccgggtcttttttcacatccagcccatgcagaccgatcggccagctcaaccagagctgccactgatcttccacacttaagcaaaccacaccagtgagtggcgaacatcaactcgtgcttgaaaaataccaacttggagcccggtttgagaagctacatcagagtctcgagatgcgacgctacaatctgcagttttcactagcttcccagtaggttgggacagttggaactctgccattgcccagcatgctgcagttcagtctgtcacccaccttgtcgatgggatttcacgtgatagccatggtggctctcttgttttcccatgtggaccatataagtgctgagacagaaatggaaggagaaggcaacgagactggcgagtgtactggctcctattactgtaagaagggggtgattttacccatttgggagccccaggacccttcctttggagacaaaattgctagagcgactgtgtattttgtggccatggtctacatgtttcttggagtctcaatcattgctgaccggttcatgtcctctatagaagtcatcacgtctcaagagaaagaaatcaccataaagaaacccaatggagagaccaccaagacaactgtgaggatctggaatgagacagtgtccaacctgaccttgatggccctggggtcttcagctccagagattctcctttcagtaatcgaggtgtgtggccataacttcactgcaggagaccttggccctagcaccatcgtggggagtgctgcattcaacatgttcatcatcattgccctttgtgtgtatgtcgtcccggatggggagacaaggaagatcaagcatctgcgtgtgttctttgtgacagcagcatggagcatctttgcctatacctggctttacatcattttgtctgtcagctcccctggggtcgtggaggtctgggaaggtttgcttactttcttcttcttccccatctgcgttgtgtttgcttgggtggcagacaggaggcttctgttttacaagtatgtctacaagaggtatcgggctggcaagcagaggggaatgattattgaacacgaaggagacaggccatcttccaagacagaaattgaaatggatgggaaagtggtcaattcccatgttgacagtttcttagatggagccctggttctggaggttgatgagagggaccaagatgatgaagaagccaggcgagaaatggctaggattctgaaggaactcaagcagaagcatccagagaaggaaatagagcaattaatagaattagccaattaccaagtcttaagtcagcagcaaaaaagtcgagcgttttaccgtattcaagctacccgcctgatgaccggagcaggcaacattttaaagaggcatgcagcagaccaagccaggaaagctgtcagcatgcatgaggtcaacacggaagtggctgaaaatgaccctgtcagtaagatcttctttgaacaagggacatatcagtgtctggagaactgtggcacagtagccctgaccattatccgcagaggtggtgatttgaccaacactgtgtttgttgacttcagaacagaggatggcacagccaatgctggatctgattacgaatttaccgaaggaactgtggtctttaagcctggtgagacccagaaggaaatcagagttggcatcattgatgatgacatctttgaggaggatgagaatttccttgtgcatctcagcaacgtcaaagtatctttggaagcctcggaagacggcatcctggaagccagtcatgtctctacccttgcttgcctgggatccccctccactgccaccgtgactatttttgatgatgaccatgctggcatctttacttttgaggaaccggtgactcatgtgagtgagagcattggcatcatggaggtgaaagttctgagaacatctggagcacgtggaaatgttatcgttccctataagaccattgaggggaccgccagaggtggaggggaggactttgaggacacatgcggagagctcgagttccagaatgacgaaattgtcaaaacaatatcagtcaaggtaattgatgatgaggagtatgagaaaaacaagaccttcttccttgagattggagagccccgcctggtggagatgagtgagaagaaagccctgttattgaatgagcttggtggcttcacaataacagggaaatacctgtatggccagcctgtcttcaggaaagttcatgctagagaacatccactcccctctactataatcaccatcgcagatgaatatgatgacaagcagccactgaccagcaaagaggaggaagagaggcgcattgcggaaatggggcgccccattctgggagagcacaccagactggaggtgatcattgaagaatcctacgagttcaagagtaccgtggacaaactgattaagaagacaaacctagccctcgtggttgggacgaacagctggagagagcagttcatcgaggcgatcactgtcagtgctggggaagatgacgatgacgacgaatgtggggaggagaagctgccctcctgttttgactacgtgatgcactttctgactgtgttctggaaggtcctcttcgcctttgtccccccgacagagtactggaacggctgggcgtgtttcatcgtctccatcctcatgatcggcctactgacggctttcattggagacctcgcttcccacttcgcctgcaccatcgccctgaaggattccgtgaccgcggtggtgttcgttgcgcttggaacctcagtgccagacacatttgcaagcaaagtggccgccacccaggaccagtatgcggatgcatccataggtaacgtcacaggcagcaacgcggtgaacgtcttcctgggcatcggtgtggcctggtccatcgccgccatctaccacgcggccaacggggaacagttcaaagtgtcccctggcacgctagctttttctgtcactctcttcaccatttttgctttcatcaatgtgggggtgctgctgtatcggcggaggccagaaattggaggtgagctgggtgggccccggactgccaagctcctcacatcctgcctctttgtgctcctgtggctcttgtacattttcttctcctccctggaggcctactgccacataaaaggcttctaaaggaacaatcagatgtagtaaatttatatatatatacatatatatatatacataaaaattatgtataatgaacagaggaaactggcatttgtcatgtccacccacctgctgatggaatccagcttcaagagcagactctgtactagggccggagagagaaggcatcacctcccgtttcccaggggcgttcgtcttgttgaaccaggcatggaggcagggccatctttacgtcagctcagcccagaagcggtgtgttctccccgggttcataaatccttaagttctttgatttgttttctgtttttgcttgttttgggtcggggtagggaggtggttgatgttagggtttggttttggttttgcagggggaagatcagggtttgtggtcctcttgtgggaggtgatgtccaatctcaatggtaaaaatggaaatcaggaagatgactctccctttgcccaaaaactttaaaaattattttggagtaagaaaggaaacgggcatggaagaagaaagaagcatgtcttcaccatattactaaatttcatgccttatctctggagtgggagcagaggtgaagtcctccctccaagaagaaacaggggagctggaatggagccaagaagagtcatggttctagatacagtctgatgtttaaagatacatcgctgcctggcacccttgttcaacaggtacaaaaacaacatgcctagattcccaggaacgcacaaagtcctttcttatctcttcagcgctggactgtgattagcaaggccctgattctgatgttctacacccgctgattccccagccctcccatcccaaaccccttctccggaccctttacccctcgtacaaacaggaagaataactccattcaaaaagcacaccatcctttccattcgcatc

SEQ ID NO: 2 - protein, 970 aa, Bos taurus
MLQFSLSPTLSMGFHVIAMVALLFSHVDHISAETEMEGEGNETGECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSTIVGSAAFNMFIIIALCVYVVPDGETRKIKHLRVFFVTAAWSIFAYTWLYIILSVSSPGVVEVWEGLLTFFFFPICVVFAWVADRRLLFYKYVYKRYRAGKQRGMIIEHEGDRPSSKTEIEMDGKVVNSHVDSFLDGALVLEVDERDQDDEEARREMARILKELKQKHPEKEIEQLIELANYQVLSQQQKSRAFYRIQATRLMTGAGNILKRHAADQARKAVSMHEVNTEVAENDPVSKIFFEQGTYQCLENCGTVALTIIRRGGDLTNTVFVDFRTEDGTANAGSDYEFTEGTVVFKPGETQKEIRVGIIDDDIFEEDENFLVHLSNVKVSLEASEDGILEASHVSTLACLGSPSTATVTIFDDDHAGIFTFEEPVTHVSESIGIMEVKVLRTSGARGNVIVPYKTIEGTARGGGEDFEDTCGELEFQNDEIVKTISVKVIDDEEYEKNKTFFLEIGEPRLVEMSEKKALLLNELGGFTITGKYLYGQPVFRKVHAREHPLPSTIITIADEYDDKQPLTSKEEEERRIAEMGRPILGEHTRLEVIIEESYEFKSTVDKLIKKTNLALVVGTNSWREQFIEAITVSAGEDDDDDECGEEKLPSCFDYVMHFLTVFWKVLFAFVPPTEYWNGWACFIVSILMIGLLTAFIGDLASHFACTIALKDSVTAVVFVALGTSVPDTFASKVAATQDQYADASIGNVTGSNAVNVFLGIGVAWSIAAIYHAANGEQFKVSPGTLAFSVTLFTIFAFINVGVLLYRRRPEIGGELGGPRTAKLLTSCLFVLLWLLYIFFSSLEAYCHIKGF